(19) United States
(12) Patent Application Publication
(10) Pub. No.: US 2026/0295211 A1
(43) Pub. Date: Oct. 1, 2026

(54) INDWELLING CATHETER ASSEMBLY AND METHOD FOR MANUFACTURING INNER NEEDLE

(71) Applicant: TERUMO KABUSHIKI KAISHA, Tokyo (JP)

(72) Inventor: Masahiro ISHIDA, Naka-gun (JP)

(73) Assignee: TERUMO KABUSHIKI KAISHA, Tokyo (JP)

(21) Appl. No.: 19/630,826

(22) Filed: Mar. 27, 2026

(30) Foreign Application Priority Data
Mar. 28, 2025 (JP) 2025-055591

(51) Int. Cl. A61M 25/00; A61M 5/158

(52) U.S. Cl. CPC A61M 25/0017; A61M 5/158; A61M 2207/00

(57) ABSTRACT

An indwelling catheter assembly includes: a catheter having a catheter lumen; and an inner needle inserted into the catheter lumen and having a distal end that protrudes from the catheter lumen. The inner needle includes: a bevel, a groove located in a side portion of the inner needle and extending along an axial direction of the inner needle to allow inflow of a fluid, and a protruding portion that is located at a distal end portion of the groove in the axial direction, is continuous with the bevel, and protrudes upward in a depth direction of the groove.

### CROSS-REFERENCE TO RELATED APPLICATIONS

This application claims priority to Japanese Patent Application No. 2025-055591, filed on Mar. 28, 2025, the entire contents of which are hereby incorporated by reference herein.

### BACKGROUND

The present disclosure relates to an indwelling catheter assembly. The present invention also relates to a method for manufacturing an inner needle of an indwelling catheter assembly.

### RELATED ART

An indwelling catheter assembly includes a catheter having a catheter lumen, and an inner needle inserted into the lumen. The distal end of the inner needle protrudes from the catheter lumen. As disclosed in JP 4280328 B2, a flashback groove is formed in the side portion of the inner needle.

Such an indwelling catheter assembly is used for giving patients infusions, for example. Specifically, the practitioner performing the infusion punctures the patient's blood vessel with the inner needle. At this point, flashback occurs. That is, the patient's blood flows into the groove. This enables the practitioner to confirm that the inner needle has reached the blood vessel. Subsequently, the practitioner moves the indwelling catheter assembly toward the patient slightly. As a result, the catheter reaches the blood vessel.

### SUMMARY

The practitioner typically determines the distance over which the indwelling catheter assembly has advanced after the inner needle reached the blood vessel based on experience. If the advancement distance of the indwelling catheter assembly is too short, the catheter may not reach the blood vessel. Conversely, if the advancement distance of the indwelling catheter assembly is too long, the distal end of the inner needle may puncture the posterior wall of the blood vessel.

Further, if biological tissue such as skin or a blood vessel enters the groove, the groove could become obstructed, reducing the likelihood of flashback occurring.

It is therefore an object of certain embodiments of the present invention to solve the above-described problems.
(1) A first embodiment of the present disclosure is an indwelling catheter assembly including a catheter having a catheter lumen; and an inner needle inserted into the catheter lumen and having a distal end that protrudes from the catheter lumen. The inner needle has, in a side portion of the inner needle, a groove extending along an axial direction of the inner needle to allow inflow of a fluid. The inner needle has, at a distal end portion of the groove in the axial direction, a protruding portion that is continuous with a bevel and that protrudes upward in a depth direction of the groove.

With this configuration, the starting point of flashback in the groove can be set closer to the catheter than in an inner needle that lacks a protruding portion. In addition, the protruding portion inhibits the groove from being obstructed by biological tissue.
(2) According to one aspect, in the indwelling catheter assembly according to (1), the protruding portion has a first surface continuous with the bevel, and a second surface continuous with the first surface via an apex of the protruding portion and extending to a bottom surface of the groove. A distal end of the first surface is located on a distal end side in the axial direction with respect to the apex, and a proximal end of the second surface is located on a proximal end side in the axial direction with respect to the apex. The first surface slopes upward in the depth direction of the groove from the distal end of the first surface toward the apex, and the second surface slopes downward in the depth direction of the groove from the apex toward the proximal end of the second surface.

This configuration can suppress damage caused by stress concentration from large, localized deformation.
(3) According to one aspect, in the indwelling catheter assembly according to (2), the groove includes a deepest portion having a maximum depth and being constant along the axial direction, and a reference depth portion having a depth 0.2% shallower than the deepest portion and being located on the second surface. A distance from the apex to the reference depth portion is 350 μm or more.

This configuration can produce an inner needle that further suppresses damage caused by large, localized deformation.
(4) According to one aspect, in the indwelling catheter assembly according to (3), the deepest portion has a depth of 90 μm or more.

With this configuration, bodily fluid readily flows into the groove (flashback).
(5) According to one aspect, in the indwelling catheter assembly according to (4), a distance from the apex to a distalmost end of the reference depth portion is 3.5 times or more the depth of the deepest portion.

In this case, an inner needle that further suppresses damage caused by large, localized deformation can be obtained.
(6) According to one aspect, in the indwelling catheter assembly according to (2), the groove has a reference depth portion with a depth 0.2% shallower than an average groove depth and being located on the second surface. A distance from the apex to the reference depth portion is 350 μm or more.

This configuration can also produce an inner needle that further suppresses damage caused by large, localized deformation.
(7) According to one aspect, in the indwelling catheter assembly according to (6), the average groove depth is an average depth of the groove from a distal end of the apex to a predetermined position on the proximal end side of the apex. The predetermined position is a position between 800 μm and 3,000 μm from the distal end of the apex.
(8) According to one aspect, in the indwelling catheter assembly according to any one of (3) to (7), a separation distance in the depth direction between the apex and an uppermost position of the groove is 90 μm or less.

With this configuration, the groove is unlikely to become obstructed by biological tissue.
(9) According to one aspect, in the indwelling catheter assembly according to any of (1) to (8), the groove has a distal end opening at a distal end of the groove in the axial direction. The distal end opening of the groove has an area of 10 μm² or greater.

With this configuration, flashback is readily produced, even when the distal end of the catheter is closer to the distal end of the inner needle than the nominal position within the tolerance range.
(10) According to one aspect, in the indwelling catheter assembly according to any of (2) to (9), the groove has a distal end opening at a distal end in the axial direction of the groove. The bevel extends along a slope direction sloped relative to the axial direction of the inner needle. A distance in the slope direction from the apex of the protruding portion to an upper end in the depth direction of the distal end opening in the groove may be 250 μm or more.

With this configuration, flashback is more readily produced, even when the distal end of the catheter is closer to the distal end of the inner needle than the nominal position within the tolerance range.
(11) A second embodiment of the present disclosure is a method of manufacturing an inner needle to be inserted into a catheter lumen of a catheter in an indwelling catheter assembly. The method includes a groove forming step to form, in a side portion of a preform that will become the inner needle, a groove extending along an axial direction of the preform to allow inflow of a fluid. In the groove forming step, a protruding portion continuous with a bevel and protruding upward in a depth direction of the groove is formed at a distal end portion of the groove in the axial direction.

This process can produce an inner needle in which the starting point of flashback is close to the catheter and that can suppress biological tissue from obstructing the groove.
(12) According to one aspect, in the method of manufacturing an inner needle according to (11), press forming may be performed in the groove forming step to press a forming mold having a groove molding portion and a protruding portion molding portion against the side portion of the preform.

With this manufacturing method, an inner needle having a groove and a protruding portion can be easily obtained.
(13) According to one aspect, in the method of manufacturing an inner needle according to (12), the forming mold may have a first sloped surface and a second sloped surface that approach each other from a proximal end toward a distal end in the axial direction in a portion of the forming mold that contacts the bevel of the preform.

With this configuration, damage to the preform caused by large, localized deformation can be avoided.

According to certain embodiments of the present disclosure, the flashback starting point of the inner needle can be set closer to the catheter. In addition, biological tissue can be suppressed from obstructing the groove that effects flashback.

### BRIEF DESCRIPTION OF THE DRAWINGS

FIG. 1 is a schematic side cross-sectional view of an indwelling catheter assembly according to an embodiment of the present invention as viewed along a width direction orthogonal to an axial direction;

FIG. 2 is a schematic side cross-sectional view of a distal end of the indwelling catheter assembly;

FIG. 3 is a schematic perspective view of the distal end of the indwelling catheter assembly;

FIG. 4 is a cross-sectional view taken along the line IV-IV in FIG. 3;

FIG. 5 is a schematic plan view of a distal end of an inner needle;

FIG. 6 is a partial perspective view illustrating a press forming operation performed by pressing a forming mold against a preform that will serve as the inner needle; and

FIG. 7A is a side view of the forming mold as viewed along the width direction orthogonal to a longitudinal direction, and FIG. 7B is a front view of the forming mold as viewed along the longitudinal direction.

### DETAILED DESCRIPTION

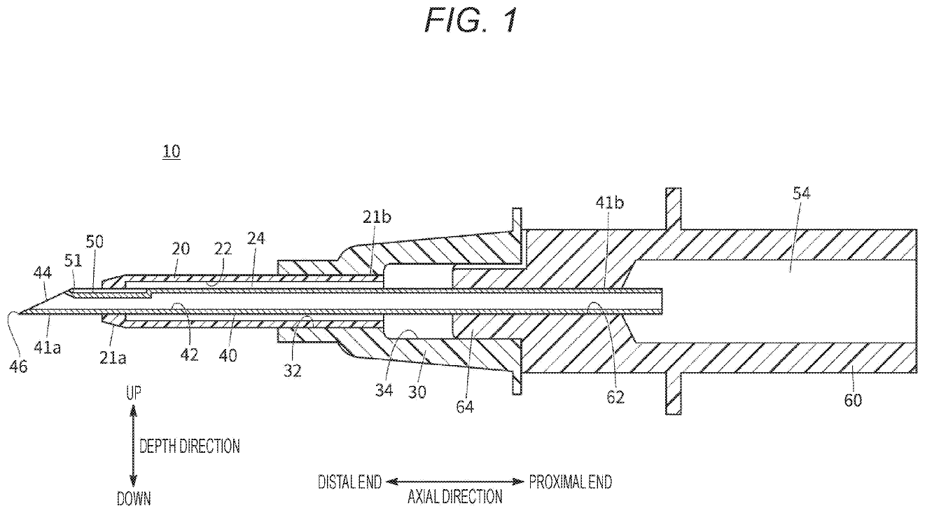

The following description illustrates embodiments in which the fluid is blood. Further, “axial direction” primarily refers to an axial direction of an inner needle 40 illustrated in FIG. 1. However, as is clear from FIG. 1, the axial direction of the inner needle 40 coincides with axial directions of a groove 50, a catheter 20, and an indwelling catheter assembly 10. Therefore, “axial direction” may also refer to the axial direction of the groove 50, catheter 20, or indwelling catheter assembly 10. A “distal end” is one end in the axial direction, and a “proximal end” is the other end in the axial direction. When the indwelling catheter assembly 10 is in use, the distal end of the indwelling catheter assembly 10 is in closer proximity to the patient than the proximal end.

A “depth direction” refers to a depth direction of the groove 50, as will be described later. The downward side in the depth direction faces the bottom of the groove 50. The upward side in the depth direction is the side opposite the downward side. A “width direction” is a direction orthogonal to both the axial direction and the depth direction.

FIG. 1 is a schematic side cross-sectional view of the indwelling catheter assembly 10 according to an embodiment of the present invention as viewed along the width direction. The indwelling catheter assembly 10 includes the catheter 20 and the inner needle 40. The catheter 20 has a catheter lumen 22. The inner needle 40 is inserted into the catheter lumen 22. A distal end portion 41a of the inner needle 40 protrudes from the catheter lumen 22. The inner needle 40 has an inner needle lumen 42. Blood flows through the inner needle lumen 42.

A proximal end portion 21b of the catheter 20 is inserted into a catheter insertion hole 32 in the distal end side of a catheter hub 30. An inner surface of the catheter insertion hole 32 is in liquid-tight contact with the outer circumferential surface of the catheter 20. The catheter hub 30 has a connection hole 34 on the proximal end side of the catheter insertion hole 32. A small-diameter portion 64 on the distal end of an inner needle hub 60 is inserted into the connection hole 34.

A proximal end portion 41b of the inner needle 40 is inserted into an inner needle insertion hole 62 in the distal end side of the inner needle hub 60. An inner surface of the inner needle insertion hole 62 is in liquid-tight contact with the outer circumferential surface of the inner needle 40. The inner needle hub 60 has an accumulation space 54 on the proximal end side of the inner needle insertion hole 62. The accumulation space 54 temporarily stores a predetermined amount of blood.

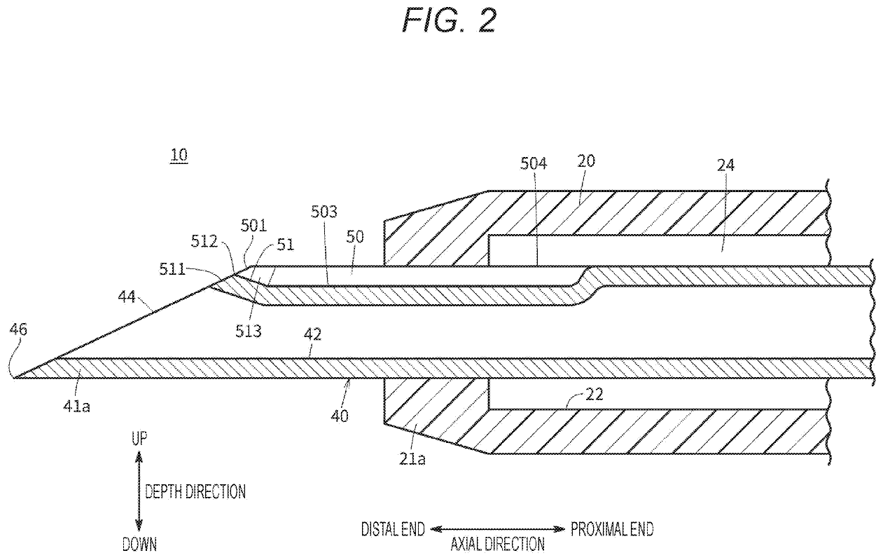

As illustrated in FIG. 2, the diameter of the catheter lumen 22 at a distalmost portion 21a of the catheter 20 (the inner diameter of the distalmost portion 21a) is slightly smaller than the inner diameter of other portions of the catheter 20 and is substantially equal to the outer diameter of the inner needle 40. Therefore, the clearance between the inner circumferential surface of the distalmost portion 21a and the outer circumferential surface of the inner needle 40 is very small. The clearance between the inner circumferential surface of the distalmost portion 21a and the outer circumferential surface of the inner needle 40 may be zero.

In the catheter 20, the diameter of the catheter lumen 22 on the proximal end side of the distalmost portion 21a is greater than the outer diameter of the inner needle 40. Consequently, an annular space 24 is formed between the outer circumferential surface of the inner needle 40 and the inner circumferential surface of the catheter lumen 22. Blood from a patient punctured by the inner needle 40 flows into the annular space 24.

While there are no particular restrictions on the material for the catheter 20, transparent resin materials, and soft resin materials in particular, are preferred. Examples include fluorine-based resins such as an ethylene tetrafluoroethylene copolymer (ETFE), polytetrafluoroethylene (PTFE), and perfluoroalkoxy fluororesin (PFA); olefin-based resins such as polyethylene and polypropylene; polyurethane-based resins such as polyurethane and polyurethane elastomers; or mixtures thereof. Examples of materials for the inner needle 40, on the other hand, include metal materials such as stainless steel, aluminum, aluminum alloys, titanium, or titanium alloys.

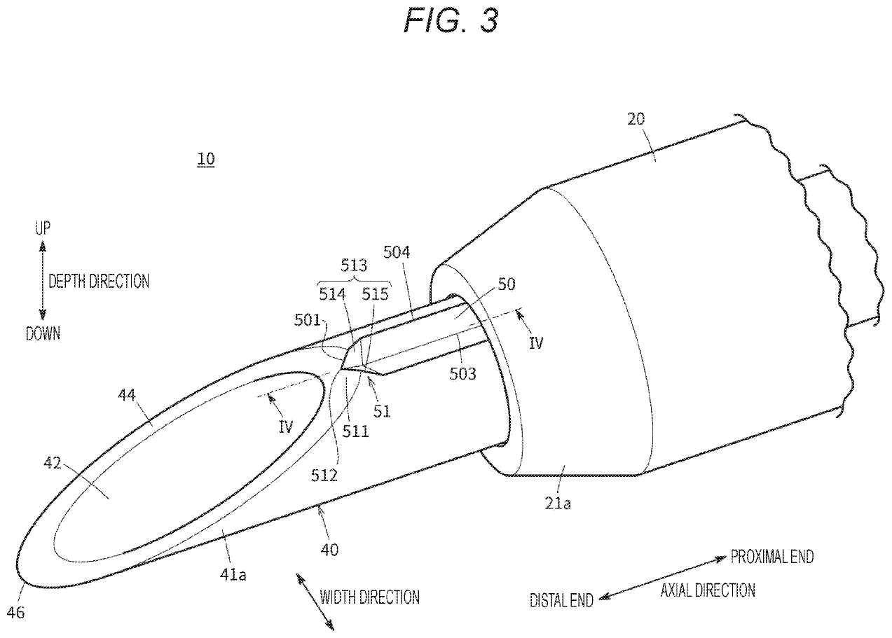

As shown in FIG. 3, the inner needle 40 has a bevel 44 as a distal end face. The bevel 44 extends so as to slope relative to the axial direction. Hereinafter, the direction in which the bevel 44 extends is defined as a “slope direction.” The inner needle 40 has a tip 46 constituting a sharp distal end of the bevel 44.

A flashback groove 50 is formed in the side portion of the inner needle 40. In the axial direction, the groove 50 is open at the bevel 44 of the inner needle 40. This opening is a distal end opening 501 of the groove 50.

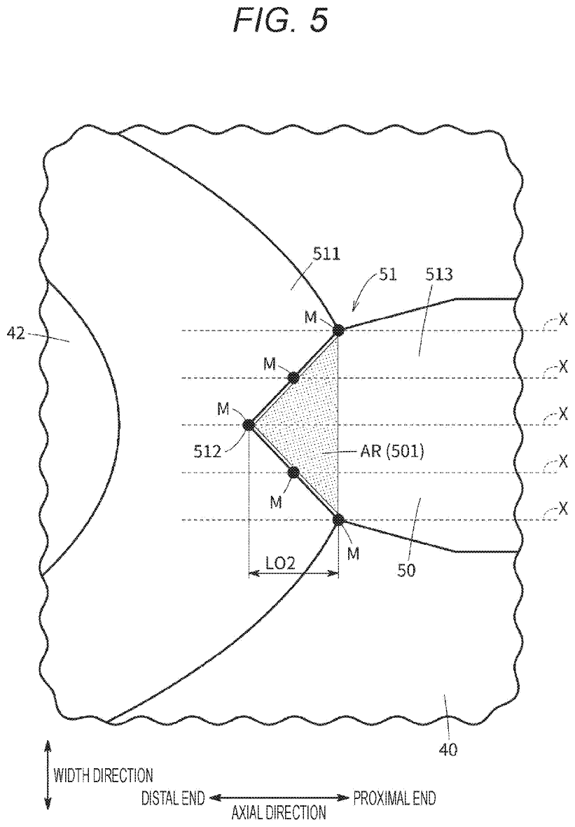

The groove 50 extends along the axial direction from the bevel 44 toward the proximal end. The groove 50 becomes narrower toward the downward side in the depth direction. That is, when a cross section of the inner needle 40 formed by cutting the inner needle 40 along a diametrical direction is viewed in the axial direction, the groove 50 has a substantially V-shape. Note that the depth direction of the groove 50 is a direction toward the bottom surface of the groove 50 orthogonal to virtual lines X extending along the axial direction (see FIG. 5).

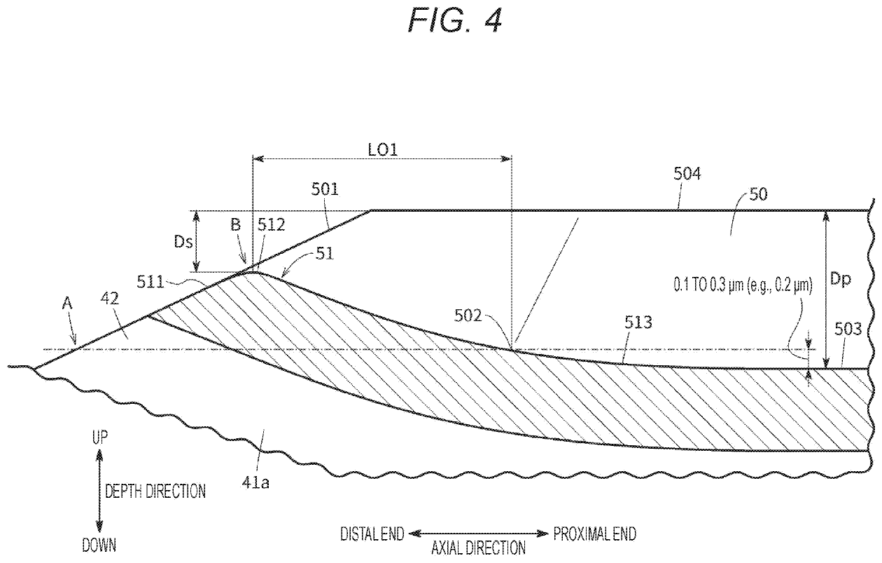

As illustrated in FIG. 4, the depth of the groove 50 changes gradually from the distal end toward the middle in the axial direction. Specifically, the depth at the distal end is less than the depth at the middle. Further, between the middle and proximal end of the groove 50, the depth is maximum and substantially constant at Dp. Hereinafter, the portion of the groove 50 having the maximum depth is defined as a deepest portion 503. As described above, the deepest portion 503 is the portion of the groove 50 between the middle and the proximal end. The depth Dp of the deepest portion 503 is preferably at least 90 μm, more preferably between 92 and 104 μm, and still more preferably between 95 and 101 μm, but may be less than 90 μm.

The groove 50 has a reference depth portion 502 in an area where the deepest portion 503 is smoothly connected to a second surface 513 of a protruding portion 51 described later. The reference depth portion 502 is located in the groove 50 on the distal end side of the deepest portion 503, and is positioned 0.1 to 0.3 μm shallower than the deepest portion 503, and preferably 0.2 μm shallower. In other words, the reference depth portion 502 is preferably located about 0.1 to 0.3% shallower, and more preferably about 0.2% shallower, with respect to the depth Dp of the deepest portion 503.

Note that the reference depth portion 502 may be based on the average groove depth rather than the deepest portion 503. In this case, the reference depth portion 502 is preferably located about 0.1 to 0.3% shallower than the average groove depth, and more preferably about 0.2% shallower. Here, the average groove depth is the average depth of the groove 50 over a range of 800 to 3,000 μm from the distal end of an apex 512 toward the proximal end.

The inner needle 40 further includes the protruding portion 51. The protruding portion 51 is located between the bevel 44 and the distal end of the groove 50 in the axial direction. The protruding portion 51 protrudes upward in the depth direction of the groove 50. As illustrated FIGS. 2 to 4, the protruding portion 51 has a first surface 511, the apex 512, and the second surface 513.

The first surface 511 of the protruding portion 51 is integrally continuous with the bevel 44. In other words, the first surface 511 is part of the bevel 44. That is, the first surface 511 is a sloped surface that slopes upward in the depth direction from the distal end toward the proximal end. Note that the first surface 511 may also be a vertical surface extending along the depth direction. In this case, the position of the first surface 511 in the axial direction is aligned with the position of the apex 512 in the axial direction.

The apex 512 is the part of the protruding portion 51 positioned farthest upward in the depth direction. The apex 512 is at a position lower than an uppermost position 504 of the groove 50 in the depth direction. Further, the apex 512 is located on the proximal end side with respect to the distal end of the first surface 511. In other words, the distal end of the first surface 511 is located on the distal end side of the apex 512.

The second surface 513 is continuous with the first surface 511 via the apex 512. In the configuration shown in FIG. 3, the second surface 513 has a first sloped portion 514 and a second sloped portion 515. The first sloped portion 514 and second sloped portion 515 approach each other while extending downward in the depth direction and also while extending from the proximal end toward the distal end in the axial direction. That is, the first sloped portion 514 and the second sloped portion 515 form a tapered shape in both the depth direction and the axial direction. However, the second surface 513 may also be a single surface.

As is clear from FIGS. 2 and 4, the proximal end of the second surface 513 is located on the proximal end side with respect to the apex 512. That is, the second surface 513 in this embodiment is a sloped surface that slopes downward in the depth direction from the distal end toward the proximal end. Note that the second surface 513 may also be a vertical surface extending along the depth direction. In this case, the position of the apex 512 in the axial direction is aligned with the position of the second surface 513 in the axial direction.

As illustrated in FIG. 4, the distance in the axial direction from the apex 512 to the reference depth portion 502 is defined as a distal end groove length LO1. The distal end groove length LO1 is preferably at least 350 μm, and more preferably at least 400 μm. The distal end groove length LO1 is still more preferably 375 to 425 μm, and even more preferably 390 to 410 μm. However, the distal end groove length LO1 may be less than 350 μm.

Further, the distal end groove length LO1 is preferably at least 3.5 times the depth Dp of the deepest portion 503, and more preferably 4.0 to 4.5 times the depth Dp. However, the distal end groove length LO1 may be less than 3.5 times the depth Dp of the deepest portion 503.

The distance of a straight line in the depth direction between the apex 512 and the uppermost position 504 of the groove 50 is defined as a separation distance Ds. The separation distance Ds is preferably no greater than 90 μm, and more preferably no greater than 85 μm. However, the separation distance Ds may be greater than 90 μm.

A shaded region AR in FIG. 5 represents the distal end opening 501 of the groove 50 in a plan view from above the groove 50 in the depth direction. The area of the distal end opening 501 is preferably at least 10 μm², and more preferably at least 12 μm². However, the area of the distal end opening 501 may be less than 10 μm².

The opening area can be found by performing the following simulation analysis using an analysis device (not shown). First, the analysis device draws a plurality of virtual lines X extending along the axial direction and passing through the apex 512 in a planar image, as illustrated in FIG. 5. The analysis device places a mark M at the intersection between each virtual line X and the apex 512. Next, the analysis device forms the region AR by connecting all the marks M and calculates the area of the region AR. The area of the region AR obtained in this manner is defined as the opening area of the distal end opening 501 of the groove 50. This allows the opening area of the distal end opening 501 to be determined accurately even when the edge of the distal end opening 501 has a slight curve.

FIG. 5 illustrates a slope-direction groove length LO2 along the bevel 44 in the distal end opening 501 of the groove 50 when viewing the inner needle 40 in a plan view. The slope-direction groove length LO2 is preferably at least 250 μm, and more preferably at least 270 μm. However, the slope-direction groove length LO2 may be less than 250 μm.

Next, a method of using the indwelling catheter assembly 10 shown in FIG. 1 will be described.

The indwelling catheter assembly 10 is used when administering (infusing) a predetermined medicinal solution into a patient's blood vessel. The practitioner punctures the patient's blood vessel with the tip 46 of the inner needle 40 in the indwelling catheter assembly 10. Once the bevel 44 has entered the blood vessel, the patient's blood flows into the inner needle hub 60 through the inner needle lumen 42. When the apex 512 subsequently enters the blood vessel, the patient's blood flows through the groove 50 from the distal end opening 501 (see FIG. 2) of the groove 50 into the catheter 20. In other words, flashback occurs in the groove 50. When the opening area of the groove 50 (see FIG. 5) is 10 μm² or greater or when the slope-direction groove length LO2 is 250 μm or greater, flashback is likely to occur, even if the distal end of the catheter 20 is closer to the distal end of the inner needle 40 than the nominal position within the tolerance range.

With an inner needle that lacks a protruding portion 51, the distal end opening 501 of the groove 50 is located in the position indicated by arrow A in FIG. 4. Therefore, the starting point of flashback in the groove 50 is also the position indicated by arrow A. In contrast, with the inner needle 40 having the protruding portion 51, the position of the distal end opening 501 (the flashback starting point) in the groove 50 is near the apex 512, as indicated by arrow B. From this, it can be seen that the starting point of flashback can be set closer to the catheter 20 in the present embodiment than in the conventional technology that uses an inner needle with no protruding portion 51.

Further, the first surface 511 of the protruding portion 51 suppresses obstruction of the groove 50 by biological tissue such as a blood vessel wall or skin. Obstruction of the groove 50 by biological tissue is further inhibited when the separation distance Ds is 90 μm or less. Further, biological tissue is less likely to obstruct the groove 50 when the depth Dp is 90 μm or more at the deepest portion 503 of the groove 50 because biological tissue has a small volume. For the reasons described above, hindrance of the progress of flashback resulting from the groove 50 being obstructed by biological tissue is suppressed.

Blood is temporarily stored in the inner needle hub 60 shown in FIG. 1. By visually confirming the blood in the inner needle hub 60, the practitioner can ascertain that the inner needle 40 has entered the blood vessel.

Thereafter, the practitioner advances the indwelling catheter assembly 10 into the patient's blood vessel. Once the apex 512 has entered the blood vessel, the patient's blood flows from the distal end opening 501 of the groove 50 (see FIG. 2), through the groove 50, and into the catheter 20. As described above, the starting point of flashback in the present embodiment is closer to the distal end of the catheter 20 than in the conventional technology (see FIG. 4).

That is, situations in which the catheter 20 is advanced an insufficient distance are avoided with the present embodiment. Therefore, situations in which the catheter 20 does not reach the blood vessel are avoided.

Subsequently, the practitioner moves the catheter 20 slightly to advance the distal end of the catheter 20 into the blood vessel.

The practitioner then holds the inner needle hub 60 (see FIG. 1) with one hand while gripping the catheter 20 with the other hand, pushes the catheter 20 in a direction toward the distal end, and removes the small-diameter portion 64 of the inner needle hub 60 from the connection hole 34 in the catheter hub 30. As a result, the catheter 20 is indwelled in the patient's blood vessel.

The practitioner then withdraws the inner needle 40 from the catheter lumen 22 of the catheter 20. Next, the practitioner connects the tube of a fluid bag (not shown) to the catheter hub 30. This allows the medicinal solution in the fluid bag to be administered to the patient via the blood vessel.

Next, a method of manufacturing the inner needle 40 will be described. The method of manufacturing the inner needle 40 includes a groove forming step. The groove 50 and the protruding portion 51 are formed in the groove forming step.

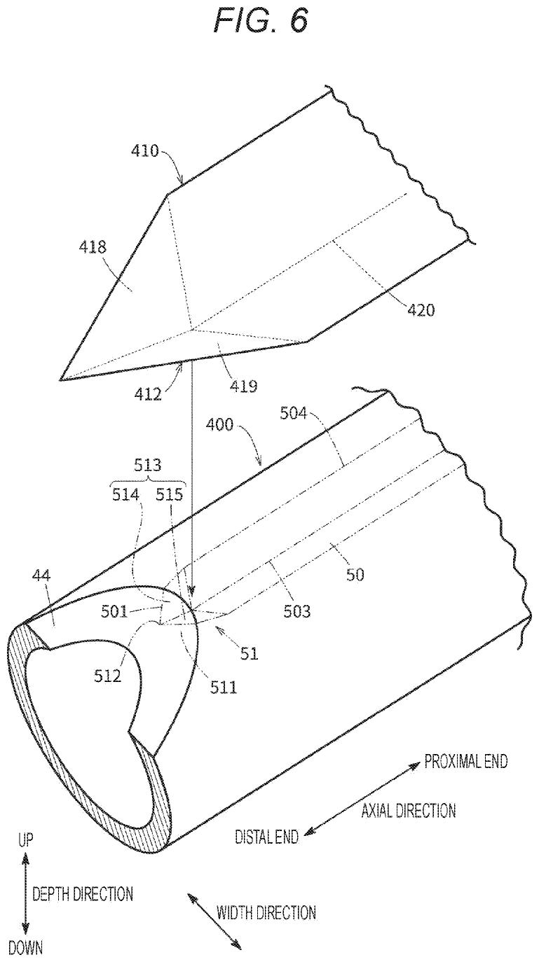

As illustrated in FIG. 6, the groove 50 and the protruding portion 51 are preferably formed by press forming a preform 400 that will become the inner needle 40. This approach is described below, but the processing method for forming the groove 50 and the protruding portion 51 is not limited to press forming. Other processing methods for forming the groove 50 and the protruding portion 51 include grinding or laser processing.

The preform 400 is configured similarly to the inner needle 40, except the preform 400 has neither the groove 50 nor the protruding portion 51. That is, the preform 400 has the tip 46 and the bevel 44.

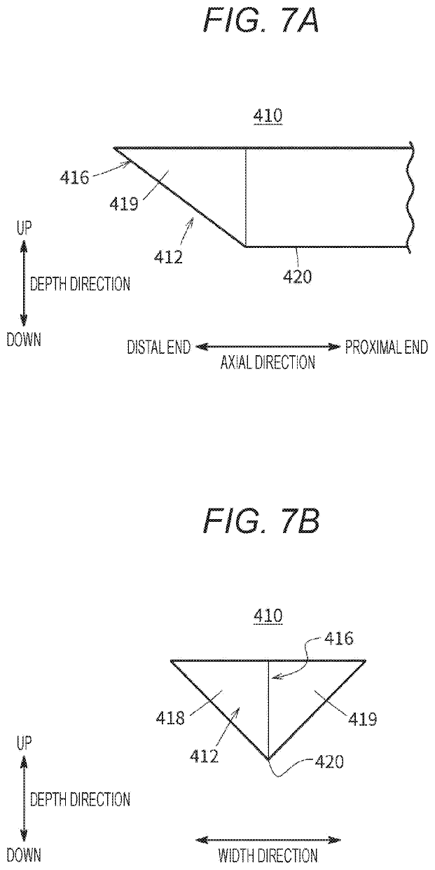

Press forming of the preform 400 is performed with a press forming apparatus (not shown) having a forming mold 410 shown in FIGS. 7A, 7B, and 6. The press forming apparatus includes a die (not shown). The preform 400 is supported in the die in advance.

As illustrated in FIGS. 7A and 7B, the forming mold 410 has a protruding portion molding portion 412, and a groove molding portion 420. As illustrated in FIG. 6, the protruding portion molding portion 412 is pressed against the distal end of the preform 400, compressing the distal end downward in the depth direction to form the distal end opening 501 of the groove 50 and the protruding portion 51. Accordingly, the protruding portion molding portion 412 has a recessed part 416 (see FIGS. 7A and 7B) for forming the apex 512 and the second surface 513 of the protruding portion 51. The recessed part 416 has a first sloped surface 418 and a second sloped surface 419. The first sloped surface 418 is the surface that forms the first sloped portion 514 of the second surface 513 (see FIG. 6). The second sloped surface 419 is the surface that forms the second sloped portion 515 of the second surface 513. Therefore, the first sloped surface 418 and second sloped surface 419 are substantially lambda (Λ) shaped (see FIGS. 7A and 7B).

As illustrated in FIG. 6, the groove molding portion 420 is pressed against the side portion of the preform 400 and compresses a mid-portion of the side portion downward in the depth direction to form the groove 50. Because the groove 50 has a substantially V-shape, the groove molding portion 420 has a substantially Λ-shape.

The operator operates the press forming apparatus while the preform 400 is supported in the die. This operation moves the forming mold 410 toward the preform 400. As can be seen in FIG. 6, this results in the protruding portion molding portion 412 and the groove molding portion 420 being pressed against the side portion of the preform 400. As the forming mold 410 moves farther, the protruding portion molding portion 412 and the groove molding portion 420 compress the side portion of the preform 400. Consequently, the groove 50 and the protruding portion 51 are formed.

As illustrated in FIG. 2, the protruding portion 51 has the first surface 511 integrated with the bevel 44, the apex 512, and the second surface 513 that is continuous with the first surface 511 via the apex 512. The first surface 511 is a sloped surface that slopes upward in the depth direction from the distal end toward the apex 512. The second surface 513 is a sloped surface that slopes downward in the depth direction from the apex 512 toward the proximal end. When a protruding portion 51 with this shape is formed simultaneously with the groove 50 during press forming, the preform 400 avoids damage resulting from stress concentration caused by large, localized deformation.

A pressing condition when forming the groove 50 and the protruding portion 51 is preferably that either the distal end groove length LO1 is greater than or equal to 350 μm or that the distal end groove length LO1 is greater than or equal to 3.5 times the depth Dp of deepest portion 503. Satisfying this condition avoids damage to part of the preform 400 (e.g., the distal end) caused by large, localized deformation. Note that the distal end groove length LO1 is more preferably 4.0 to 4.5 times the depth Dp of the deepest portion 503.

Further, the second surface 513 of the protruding portion 51 is formed as a surface having the first sloped portion 514 and the second sloped portion 515 that form a tapered shape. This further avoids damage to part of the preform 400 (e.g., the distal end).

That is, press forming performed on the preform 400 under the above condition can produce an inner needle 40 in which damage caused by large, localized deformation is suppressed.

The effects of the present embodiment are summarized as follows.

As illustrated in FIGS. 2 to 4, the inner needle 40 of the indwelling catheter assembly 10 has the protruding portion 51 between the bevel 44 and the distal end of the groove 50 in the axial direction. The protruding portion 51 protrudes upward in the depth direction of the groove 50.

The starting point of flashback in the groove 50 can be set closer to the catheter 20 than in an inner needle 40 that lacks a protruding portion 51. Therefore, the distance that the catheter 20 advances after the inner needle 40 punctures the patient can be reduced from that in the conventional technology. This can avoid situations in which the catheter 20 is not sufficiently advanced to reach the blood vessel.

Further, by serving as a barrier, the protruding portion 51 suppresses biological tissue such as a blood vessel or skin from obstructing the groove 50. Therefore, hindrance of flashback in the groove 50 resulting from biological tissue obstructing the groove 50 is avoided.

The protruding portion 51 has the first surface 511 integrated with the bevel 44, the apex 512, and the second surface 513 continuous with the first surface 511 via the apex 512. The apex 512 is the uppermost part of the protruding portion 51 in the depth direction of the groove 50. The first surface 511 is a sloped surface that slopes upward in the depth direction from the distal end toward the apex 512. The second surface 513 is a sloped surface that slopes downward in the depth direction from the apex 512 toward the proximal end.

By simultaneously forming the protruding portion 51 having such a shape when forming the groove 50 in the side portion of the inner needle 40 during press forming, an inner needle 40 can be obtained in which damage caused by large, localized deformation is suppressed.

As illustrated in FIG. 4, the groove 50 has the reference depth portion 502 in an area where the deepest portion 503 is smoothly connected to the second surface 513 of the protruding portion 51. The reference depth portion 502 is located at a position 0.1 to 0.3 μm shallower than the deepest portion 503, and preferably 0.2 μm shallower. In other words, the reference depth portion 502 is preferably located about 0.1 to 0.3% shallower, and more preferably about 0.2% shallower, with respect to the depth Dp of the deepest portion 503. The distal end groove length LO1, which is the distance from the apex 512 to the reference depth portion 502, is at least 350 μm. This arrangement can also produce an inner needle 40 in which damage is suppressed. The reference depth portion 502 may be located at a position shallower than the average groove depth. In this case, the reference depth portion 502 is preferably located about 0.1 to 0.3% shallower than the average groove depth, and more preferably about 0.2% shallower.

The depth Dp of the deepest portion 503 is at least 90 μm. With this configuration, flashback readily occurs in the groove 50.

The separation distance Ds in the depth direction between the apex 512 and the uppermost position 504 of the groove 50 is 90 μm or less. With this configuration, the groove 50 is unlikely to become obstructed by biological tissue.

The groove 50 has the distal end opening 501. The area of the region AR shown in FIG. 5 (the area of the distal end opening 501) is at least 10 μm². In this case, flashback is readily produced in the groove 50, even when the distal end of the catheter 20 is closer to the distal end of the inner needle 40 than the nominal position within the tolerance range.

In the extended direction of the bevel 44 (a slope direction sloped relative to the axial direction of the inner needle 40), the slope-direction groove length LO2 is at least 250 μm.

With this configuration, flashback occurs more readily in the groove 50, even when the distal end of the catheter 20 is close to the distal end of the inner needle 40 within the tolerance range.

As illustrated in FIG. 6, the method of manufacturing the inner needle 40 includes the groove forming step. The groove 50 and the protruding portion 51 are formed in the preform 400 in the groove forming step. Accordingly, the inner needle 40 described above can be efficiently produced.

In the groove forming step, the preform 400 is subjected to press forming. Specifically, the groove molding portion 420 and the protruding portion molding portion 412 of the forming mold 410 (see FIGS. 7A and 7B) are pressed against the preform 400. As a result, an inner needle 40 having the groove 50 and the protruding portion 51 can be easily obtained.

As illustrated in FIG. 6, the forming mold 410 has the first sloped surface 418 and the second sloped surface 419 in an area that contacts the bevel 44 of the preform 400. The first sloped surface 418 and second sloped surface 419 approach each other from the proximal end toward the distal end in the axial direction. With this configuration, damage to the preform 400 (the inner needle 40) caused by stress concentration from large, localized deformation can be avoided when performing the groove forming step.

Although embodiments of the present disclosure have been described in detail, this invention is not limited to the embodiments described above. Various additions, substitutions, modifications, partial deletions, and the like may be made to these embodiments without departing from the gist of the present disclosure or without departing from the spirit of the present invention derived from the contents described in the claims and equivalents thereof. Further, the embodiments can be implemented in combination. For example, the order of each operation and each step in the above embodiments is provided as an example, and the present invention is not limited thereto. The same applies when numerical values or expressions are used in the description of the above embodiments.

What is claimed is:

1. An indwelling catheter assembly comprising:
a catheter having a catheter lumen; and
an inner needle inserted into the catheter lumen and having a distal end that protrudes from the catheter lumen,
wherein the inner needle comprises:
a bevel,
a groove located in a side portion of the inner needle and extending along an axial direction of the inner needle to allow inflow of a fluid, and
a protruding portion that is located at a distal end portion of the groove in the axial direction, is continuous with the bevel, and protrudes upward in a depth direction of the groove.

2. The indwelling catheter assembly according to claim 1, wherein:
the protruding portion has a first surface continuous with the bevel, and a second surface continuous with the first surface via an apex of the protruding portion and extending to a bottom surface of the groove,
a distal end of the first surface is located on a distal end side in the axial direction with respect to the apex, and a proximal end of the second surface is located on a proximal end side in the axial direction with respect to the apex,
and the first surface slopes upward in the depth direction of the groove from the distal end of the first surface toward the apex, and the second surface slopes downward in the depth direction of the groove from the apex toward the proximal end of the second surface.

3. The indwelling catheter assembly according to claim 2, wherein:
the groove includes a deepest portion having a maximum depth and being constant along the axial direction, and a reference depth portion having a depth 0.2% shallower than the deepest portion and located on the second surface, and
a distance from the apex to the reference depth portion is 350 μm or more.

4. The indwelling catheter assembly according to claim 3, wherein the deepest portion has a depth of 90 μm or more.

5. The indwelling catheter assembly according to claim 4, wherein a distance from the apex to a distalmost end of the reference depth portion is 3.5 times or more the depth of the deepest portion.

6. The indwelling catheter assembly according to claim 2, wherein:
the groove has a reference depth portion with a depth 0.2% shallower than an average groove depth and located on the second surface, and
a distance from the apex to the reference depth portion is 350 μm or more.

7. The indwelling catheter assembly according to claim 6, wherein the average groove depth is an average depth of the groove from a distal end of the apex to a predetermined position on the proximal end side of the apex, and the predetermined position is a position between 800 μm and 3,000 μm from the distal end of the apex.

8. The indwelling catheter assembly according to claim 3, wherein a separation distance in the depth direction between the apex and an uppermost position of the groove is 90 μm or less.

9. The indwelling catheter assembly according to claim 1, wherein the groove has a distal end opening at a distal end of the groove in the axial direction, and the distal end opening of the groove has an area of 10 μm² or greater.

10. The indwelling catheter assembly according to claim 2, wherein the groove has a distal end opening at a distal end in the axial direction of the groove, the bevel extends along a slope direction sloped relative to the axial direction of the inner needle, and a distance in the slope direction from the apex of the protruding portion to an upper end in the depth direction of the distal end opening in the groove is 250 μm or more.

11. A needle for an indwelling catheter assembly, the needle comprising:
a bevel;
a groove located in a side portion of the needle and extending along an axial direction of the needle to allow inflow of a fluid; and
a protruding portion that is located at a distal end portion of the groove in the axial direction, is continuous with the bevel, and protrudes upward in a depth direction of the groove.

12. The needle according to claim 11, wherein:
the protruding portion has a first surface continuous with the bevel, and a second surface continuous with the first surface via an apex of the protruding portion and extending to a bottom surface of the groove,
a distal end of the first surface is located on a distal end side in the axial direction with respect to the apex, and a proximal end of the second surface is located on a proximal end side in the axial direction with respect to the apex,
and the first surface slopes upward in the depth direction of the groove from the distal end of the first surface toward the apex, and the second surface slopes downward in the depth direction of the groove from the apex toward the proximal end of the second surface.

13. The needle according to claim 12, wherein:
the groove includes a deepest portion having a maximum depth and being constant along the axial direction, and a reference depth portion having a depth 0.2% shallower than the deepest portion and located on the second surface, and
a distance from the apex to the reference depth portion is 350 μm or more.

14. The needle according to claim 13, wherein the deepest portion has a depth of 90 μm or more.

15. The needle according to claim 14, wherein a distance from the apex to a distalmost end of the reference depth portion is 3.5 times or more the depth of the deepest portion.

16. The needle according to claim 12, wherein:
the groove has a reference depth portion with a depth 0.2% shallower than an average groove depth and located on the second surface, and
a distance from the apex to the reference depth portion is 350 μm or more.

17. The needle according to claim 16, wherein the average groove depth is an average depth of the groove from a distal end of the apex to a predetermined position on the proximal end side of the apex, and the predetermined position is a position between 800 μm and 3,000 μm from the distal end of the apex.

18. A method of manufacturing an inner needle to be inserted into a catheter lumen of a catheter in an indwelling catheter assembly, the method comprising:
a groove forming step comprising:
forming, in a side portion of a preform that will become the inner needle, a groove extending along an axial direction of the preform to allow inflow of a fluid, and
forming, at a distal end portion of the groove in the axial direction, a protruding portion continuous with a bevel of the inner needle and protruding upward in a depth direction of the groove.

19. The method of manufacturing an inner needle according to claim 18, wherein, the groove forming step comprises press forming to press a forming mold having a groove molding portion and a protruding portion molding portion against the side portion of the preform.

20. The method of manufacturing an inner needle according to claim 19, wherein the forming mold has a first sloped surface and a second sloped surface that approach each other from a proximal end toward a distal end in the axial direction in a portion of the forming mold that contacts the bevel of the preform.
